(19) United States
(12) Patent Application Publication
(10) Pub. No.: US 2011/0076729 A1
(43) Pub. Date: Mar. 31, 2011

(54) METHODS OF MAKING LOW MOLECULAR WEIGHT HEPARIN COMPOSITIONS

(75) Inventors: Zainab Sirajbhai Mamuwala, Lowell, MA (US); Ganesh Venkataraman, Bedford, MA (US)

(73) Assignee: MOMENTA PHARMACEUTICALS, INC., Cambridge, MA (US)

(21) Appl. No.: 12/864,660

(22) PCT Filed: Feb. 19, 2009
(86) PCT No.: PCT/US09/34493

§ 371 Date: Nov. 23, 2010

Related U.S. Application Data
(60) Provisional application No. 61/030,069, filed on Feb. 20, 2008.

(51) Int. Cl. C12P 19/04; C08B 37/10

(52) U.S. Cl. 435/101; 536/21

(57) ABSTRACT

Methods of making LMWH compositions are provided to provide the LMWH compositions at a yield of at least about 10%.

This application claims priority from 61/030,069, filed Feb. 20, 2008, hereby incorporated by reference.

### BACKGROUND

Coagulation is a physiological pathway involved in maintaining normal blood hemostasis in mammals. Under conditions in which a vascular injury occurs, the coagulation pathway is stimulated to form a blood clot to prevent the loss of blood. Immediately after the vascular injury occurs, blood platelets begin to aggregate at the site of injury forming a physical plug to stop the leakage. In addition, the injured vessel undergoes vasoconstriction to reduce the blood flow to the area and fibrin begins to aggregate forming an insoluble network or clot, which covers the ruptured area.

When an imbalance in the coagulation pathway shifts towards excessive coagulation, the result is the development of thrombotic tendencies, which are often manifested as heart attacks, strokes, deep vein thrombosis, and acute coronary syndromes such as myocardial infarcts, and unstable angina. Furthermore, an embolism can break off from a thrombus and result in a pulmonary embolism or cerebral vascular embolism including stroke or transient ischemia attack. Current therapies for treating disorders associated with imbalances in the coagulation pathway involve many risks and must be carefully controlled.

Heparin and low molecular weight heparins (LMWHs), complex, sulfated polysaccharides isolated from endogenous sources, are potent modulators of hemostasis. Heparin, a highly sulfated heparin-like glycosaminoglycan (HLGAG) produced by mast cells, is a widely used clinical anticoagulant, and is one of the first biopolymeric drugs and one of the few carbohydrate drugs. Heparin and molecules derived from it are potent anticoagulants that are used in a variety of clinical situations, especially for thromboembolic disorders including the prophylaxis and treatment of deep venous thrombosis and pulmonary embolism, arterial thromboses, and acute coronary syndromes like myocardial infarction and unstable angina. Heparin and LMWHs interact with multiple components of the coagulation cascade to inhibit the clotting process. Heparin primarily elicits its effect through two mechanisms, both of which involve binding of antithrombin III (AT-III) to a specific pentasaccharide sequence, H_{NAc/S,6S}GH_{NS,3S,6S}I_{2S}H_{NS,6S} contained within the polymer. First, AT-III binding to the pentasaccharide induces a conformational change in the protein that mediates its inhibition of factor Xa. Second, thrombin (factor IIa) also binds to heparin at a site proximate to the pentasaccharide/AT-III binding site. Formation of a ternary complex between AT-III, thrombin and heparin results in inactivation of thrombin. Unlike its anti-Xa activity that requires only the AT-III pentasaccharide-binding site, heparin's anti-IIa activity is size-dependent, in addition to the pentasaccharide unit responsible for anti-Xa activity for the efficient formation of an AT-III, thrombin, and heparin ternary complex. Heparin also mediates the release of tissue factor pathway inhibitor (TFPI) from endothelial cells. TFPI, a heparin cofactor, is a serine protease that directly binds to and inhibits factor X. TFPI is a potent anti-thrombotic, particularly when co-administered with heparin.

Although heparin is highly efficacious in a variety of clinical situations and has the potential to be used in many others, the side effects associated with heparin therapy are many and varied. Anti-coagulation has been the primary clinical application for unfractionated heparin (UFH) for over 65 years. Due to its erratic intravenous pharmacokinetics and lack of subcutaneous bioavailability, UFH has been administered by intravenous injection instead. Additionally, the application of UFH as an anticoagulant has been hampered by the many side effects associated with non-specific plasma protein binding with UFH.

This has led to the explosion in the generation and utilization of low molecular weight heparin (LMWH) as an efficacious alternative to UFH. LMWH provide a more predictable pharmacological action, reduced side effects, and better bioavailability than UFH. Since the commercially available LMWH preparations are not fully neutralized by protamine, an unexpected reaction could have extremely adverse effects; the anti-Xa activity of enoxaparin and other LMWH are neutralizable only to an extent of about 40% with ≦2 mg Protamine/100 IU anti-Xa LMWH. The anti-IIa activity is neutralizable only to an extent of about 60% with ≦2 mg Protamine/100 IU anti-Xa LMWH. (On the other hand, the anti-Xa and anti-IIa activity of UFH is neutralizable almost completely (>90%) with ≦2 mg Protamine sulfate/100 IU anti-Xa UFH.)

Pharmaceutical preparations of these polysaccharides, typically isolated from porcine intestinal mucosa, are heterogeneous in length and composition. As such, only a portion of a typical preparation possesses anticoagulant activity. At best, the majority of the polysaccharide chains in a pharmaceutical preparation of heparin or LMWH are inactive, at worst, these chains interact nonspecifically with plasma proteins to elicit the side effects associated with heparin therapy. Therefore, it is important to develop novel LMWHs that retain the anticoagulant activity and other desired activities of UFH but have reduced side effects. LMWHs, essentially due to their reduced chains sizes and dispersity, display markedly less non-specific plasma protein binding. However, all LMWHs that are currently clinically available also possess reduced anti-IIa activity as compared to UFH. Because of this decreased activity, a larger dose of LMWH is required (compared to UFH) in order to achieve a similar anti-Xa and anti-IIa activity, and the standard tests for UFH activity, activated partial thromboplastin time (aPTT) or activated clotting time (ACT), are not useful as they rely primarily on anti-IIa activity for a readout. The most widely used test for monitoring LMWH levels is an anti-Xa activity test, which depends on the subject having sufficient levels of antithrombin III (ATIII), which is not always the case. This test is quite costly (well over $100.00) and is not routine or readily available, as samples generally must be sent to an outside lab for analysis. Consequently, the use of LMWHs so far has been largely limited to the prevention of thrombosis and not to their treatment, and the population of patients to whom it can be administered has been limited, excluding, among others, pediatric patients, patients with abnormal renal function as measured by RFI, urea, creatinine, phosphorus, glomerular filtration rate (GFR), or BUN (Blood Urea Nitrogen level) in blood and urine and the interventional cardiology patient population.

### SUMMARY OF THE INVENTION

The invention is based, in part, on methods of making LMWH compositions. The methods can produce yields of at least about 10%, for example, 15%, 20%, 30%, 40%, 50%, 60%, 70%, 80%, 90% or more of the LMWH composition relative to the amount of UFH starting material.

Accordingly, in one aspect, the invention features a method of making a LMWH composition, e.g., a LMWH composition described herein. The method includes providing an UFH composition, and digesting the UFH composition with an enzyme and/or chemical that cleaves glycosidic linkages of unsulfated uronic acids to provide a digested composition. The method further includes fractionating the digested composition, e.g., fractionating by charge or by size (e.g., using ion exchange chromatography or size exclusion chromatography), to provide the LMWH composition, e.g., a LMWH composition described herein. A yield of the LMWH composition produced by the method is at least about 10%.

In another aspect, the invention features a method of processing an UFH composition to provide a LMWH composition at a yield of at least about 10%. The method includes digesting the UFH composition with an enzyme and/or chemical that cleaves glycosidic linkages of unsulfated uronic acids and fractionating the digested composition to provide a LMWH composition described herein. The UFH composition is processed to provide a LMWH composition at a yield of at least about 10%.

In another aspect, the invention features a method of making a LMWH composition. The method includes providing a composition made by digesting an UFH composition with an enzyme and/or chemical that cleaves glycosidic linkages of unsulfated uronic acids, and subjecting the composition to one or more fractionation steps to thereby make the LMWH composition. The yield of the fractionated LMWH composition produced by the method is at least about two-fold, three-fold, four-fold or five-fold greater than that of a method that comprises fractionation, e.g., precipitation, of the UFH composition prior to digestion with the same enzyme and/or chemical.

The yield of the LMWH composition achieved by any of the methods described herein can be at least about 10%, 15% 20%, 30%, 40%, 50%, 60%, 70%, 80%, 90% or greater.

In one embodiment of the methods described herein, the yield of the purified LMWH composition produced by the method is at least about 2-fold, 3-fold, 4-fold, 5-fold, 10-fold, 15-fold, 20-fold or greater than that of a method that includes fractionation, e.g., precipitation, of the UFH composition prior to digestion with the same enzyme and/or chemical.

In one embodiment, the enzyme (or enzymes) used for digestion cleaves at one or more glycosidic linkages of unsulfated uronic acids, e.g., adjacent to an N-acetyl glucosamine residue. Examples of enzymes that can be used include, e.g., heparinase III, mutants of heparinase III, e.g., a heparinase III mutant described in U.S. Pat. No. 5,896,789 (e.g., a mutant of heparinase III having one or more histidine residue selected from the group consisting of His 36, His105, His110, His139, His152, His225, His234, His241, His424, His469, and His539 has been substituted with an alanine), heparinase II, mutants of heparinase II and heparin sulfate glycosaminoglycan lyase III from Bacteroides thetaiotaomicron. In a preferred embodiment, the enzyme used for digestion is a mutated heparinase III having an alanine at residue 225 of the amino acid sequence substituted with an alanine (MO11). In one embodiment, the enzyme may be present at a concentration in a range of about 25 to 500 units per gram of UFH, e.g., 100 to 400 or 200 to 350 units per gram of UFH. In one embodiment, for example, the enzyme may be present at a concentration of about 100 units per gram of UFH.

In one embodiment, the chemical (or chemicals) used for digestion cleaves at one or more glycosidic linkages of unsulfated uronic acids, e.g., adjacent to an N-acetyl glucosamine residue. Examples of chemicals include chemicals that cleave by oxidation (e.g., hydrogen peroxide), deamination (e.g., nitrous oxide) or beta elimination (e.g., quaternary ammonium).

Other reactants or compositions may be present or added to the digestion mixture in addition to the enzyme and/or chemical.

In one embodiment of the methods described herein, the UFH composition is digested at a temperature of about 25° C. to about 50° C. The UFH composition can be digested, e.g., at a pH level of about 5 to about 9. A salt buffer, e.g., a monovalent or divalent salt buffer, can be used with the enzyme and/or chemical to digest the UFH composition. The buffer can be, e.g., a BisTris buffer, a phosphate buffer, or an acetate buffer. For example, the salt buffer can be a monovalent or divalent acetate buffer, e.g., a sodium acetate buffer. In one embodiment, the UFH composition is digested to completion, e.g., as indicated by a UV plateau, to provide the LMWH composition.

In one embodiment of the disclosed methods, the UFH composition is not size fractionated prior to digestion with the enzyme and/or chemical. In one embodiment, the UFH composition is not subjected to a precipitation prior to digestion with the enzyme and/or chemical.

In one embodiment, the digested and fractionated composition is the drug substance. In other embodiments, the method can include one or more additional processing steps to obtain a drug substance. For example, the methods can further include subjecting the LMWH composition to one or more purification steps, e.g., filtration and/or precipitation.

In one embodiment, the method further includes subjecting the LMWH composition to one or more purification steps. In one embodiment of the method, the one or more purification steps include a filtration step.

In one embodiment, the methods include drying the LMWH composition. For example, the LMWH compositions can be dried by lyophilization, spray drying, or a drum dryer operation. In other embodiments, the dried LMWH composition is reconstituted to provide a drug product

In one embodiment, a precursor LMWH composition is provided (e.g., an intermediate composition from a method described herein) having an average chain length of about 10 to 22 disaccharides, e.g., 10 to 20 disaccharides. The precursor LMWH composition is processed, e.g., fractionated to obtain a LMWH having an average chain length of about 10 to 19 disaccharides. In one embodiment, the precursor LMWH is obtained by a method including enzymatic digestion of an UFH composition, e.g., an UFH composition that has not been fractionated and/or precipitated.

In one embodiment, the method includes digesting the UFH composition using an enzyme and/or chemical that cleaves glycosidic linkages of unsulfated uronic acids, e.g., an enzyme described herein, e.g., in aqueous buffer, e.g., in aqueous salt buffer, e.g., a sodium acetate buffer, pH of about 5-9, e.g., 7-8, at 25° C. to 52° C., e.g., 37° C., to produce a precursor LMWH composition, wherein the precursor LMWH preferably has a average chain length of 10 to 22 disaccharides, e.g., 10 to 20 disaccharides;

removing high molecular weight components from the precursor LMWH composition by a size or charge based step or steps, e.g., SEC or IEX, to produce a LMWH composition having an average chain length of 10 to 19 disaccharides; and optionally

dissolving the LMWH composition in purified water, filtering, e.g., through a 0.2 pm filter, and drying, e.g., lyophilizing, spray drying or drum drying, to obtain drug substance.

The methods described herein can further include one or more of the following steps: determining the weight average molecular weight of the LMWH composition; determining the anti-IIa activity of the LMWH composition; determining the anti-Xa activity of the LMWH composition; determining the anti-Xa activity to anti-Ha activity ratio of the LMWH composition.

In one embodiment, a method described herein provides a LWMH composition having one or more desired properties or characteristics. For example, a LMWH composition produced by the methods described herein can have one or more of the following characteristics: 100 to 400 IU/mg of Xa activity, an anti-IIa activity that is at least 50%, 60%, 70%, 80%, 90%, 95% or 100% neutralizable with protamine, e.g., as measured by activated partial thrombostatin time (ACT) or activated partial thromboplastin time (aPTT), and about a 1:1 ratio of anti-Xa activity to anti-IIa activity over time.

In one embodiment, a method described herein provides a LMWH composition having: a weight average molecular weight of about 5000 to 9500 Da, e.g., about 5000 to 8300 Da, e.g., about 5500 to 8000 Da, e.g., about 5700 to 7900 Da, e.g., about 5800 to 6800 Da; and

an anti-IIa activity of about 50 to 300, e.g., about 70 to 280, e.g., about 90 to 250 IU/mg, e.g., about 100 to 250 IU/mg, e.g., about 100 to 140 IU/mg, 150 to 200 IU/mg, about 130 to 190 IU/mg, e.g., about 155 to 195 IU/mg.

In one embodiment, a method described herein provides a LMWH composition having:

a weight average molecular weight of about 5000 to 9500 Da, e.g., about 5000 to 8300 Da, e.g., about 5000 to 8000 Da, about 5500 to 8000 Da, e.g., about 5700 to 7900 Da, e.g., about 5800 to 6800 Da; and

anti-IIa activity that is at least 50%, 60%, 70%, 80%, 85%, 90%, 95%, 99% or 100% neutralizable with protamine, e.g., as measured by ACT or aPTT. Preferably, the anti-IIa activity of the LMWH is neutralized by at least 50%, 60%, 70%, 80%, 85%, 90%, 95%, 99% or 100% within 5, 10, 15, 30 minutes after protamine administration.

In one embodiment, a method described herein provides a LMWH composition having:

a weight average molecular weight of about 5000 to 9500 Da, e.g., about 5000 to 8300 Da, e.g., about 5500 to 8000 Da, e.g., about 5700 to 7900 Da, e.g., about 5800 to 6800 Da; and

ΔUH_{NAc,6S}GH_{NS,3S,6S} is 5 to 15%, e.g., 7 to 14%, e.g., 9 to 12%, of the composition, e.g., as measured by capillary electrophoresis (CE) as a mole %. Preferably the ΔUH_{NAc,6S}GH_{NS,3S,6S} is about 5 to 15%, e.g., 7 to 14%, e.g., 9 to 12%, of the chains in the composition, e.g., as measured by CE as a mole %.

In one embodiment, a method described herein provides a LMWH composition having:

an average chain length of about 8 to 19 disaccharides; and

ΔUH_{NAc,6S}GH_{NS,3S,6S} is 5 to 15%, e.g., 7 to 14%, e.g., 9 to 12%, of the composition, e.g., as measured by CE as a mole %. Preferably the ΔUH_{NAc,6S}GH_{NS,3S,6S} is about 5 to 15%, e.g., 7 to 14%, e.g., 9 to 12%, of the chains in the composition, e.g., as measured by CE as a mole %.

In one embodiment, a method described herein provides a LMWH composition having:

a weight average molecular weight of 5000 to 9500 Da, e.g., about 5000 to 8300 Da, e.g., about 5500 to 8000 Da, e.g., about 5700 to 7900 Da, e.g., about 5800 to 6800 Da; and

an anti Xa to anti-IIa ratio of 3:1 or less, e.g., 2:1, e.g., 1.6:1, 1.5:1, 1.4:1, 1.3:1, 1.2:1, 1.1:1, 1:1 or 0.5:1.

In one embodiment, a method described herein provides a LMWH composition having one or more of the following characteristics:

the composition has substantially no (e.g., at least 85%, 90%, 95% or more of the chains do not have) modified reducing end structures; at least 60%, 70%, 80%, 85%, 90%, 95%, 99% of the chains of the composition have H_{NAc} at the reducing end; less than 90%, 95%, 98%, 99%, preferably none of the chains of the composition have a sulfated ΔU at the non-reducing end; and there is substantially no linkage region (e.g., less than 0.1% linkage region) present in the composition.

In one embodiment, a manufactured LMWH composition has two, three or all of these characteristics.

In one embodiment, a method described herein provides a LMWH composition having the following structure:

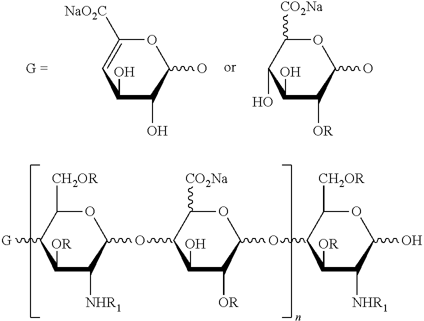

wherein R is H or SO₃X;

R1 is SO₃X or COCH₃;

X is a monovalent or divalent cation;

n=2-50, e.g., 2-40; and

the composition preferably has an average value for n of 8 to 19 (e.g., 9-16, 8-16, 8-15). In a preferred embodiment, 5 to 15% of the chains in the LMWH composition have ΔUH_{NAc,6S}GH_{NS,3S,6S}, e.g., as measured by CE as a mole %, e.g., 5 to 15% of the chains in the LMWH composition have ΔUH_{NAc,6S}GH_{NS,3S,6S}, at the non-reducing end of the molecule.

In one embodiment, the LMWH composition has the following structure:

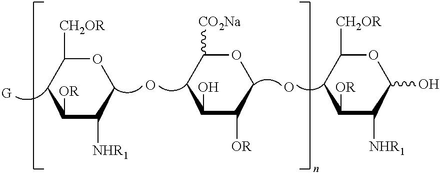

wherein:

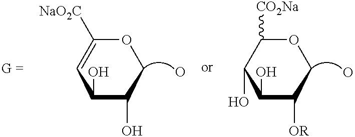

R is H or SO₃X;

R1 is SO₃X or COCH₃;

X is a monovalent or divalent cation;

n=2-50, e.g., 2-40; and

the composition preferably has an average value for n of 10 to 19.

Any of the LMWH compositions described herein, e.g., described above, can have other properties. E.g., one of the above described compositions can further have one or more of functional or structural properties set out below.

Thus, in one embodiment, a LMWH composition produced has an anti-Xa activity of about 100 to 400 IU/mg, e.g., about 120 to 380 IU/mg, e.g., about 150 to 350 IU/mg, e.g., about 170 to 330 IU/mg, e.g., about 180 to 300 IU/mg, e.g., about 150 to 200 IU/mg, 200 to 300 IU/mg, 130 to 220 IU/mg, 225 to 274 IU/mg.

In one embodiment, a LMWH composition has an anti-Xa activity that is at least 50%, 60%, 70%, 80%, 85%, 90%, 95%, 99%, 100% neutralizable with protamine, e.g., as measured by anti-Xa activity, ACT or aPTT. Preferably, the anti-Xa activity is neutralized by at least 50%, 60%, 70%, 80%, 85%, 90%, 95%, 99% or 100% within 5, 10, 15 minutes after protamine administration. For example, the anti-Xa activity can be neutralized by at least 50%, 60%, 70%, 80%, 85%, 90%, 95%, 99% or 100% within 5, 10, 15, 30 minutes after protamine administration at a dose of about 1, 2, 3 mg of the LMWH composition per 100 anti-Xa IU of plasma.

In one embodiment, a method described herein produces a LMWH composition having one or more of the following properties: the activity of the composition can be monitored by aPTT and/or ACT; the polydispersity of the composition is less than 1.6, e.g., the polydispersity is about 1.6 to 1.1, e.g., 1.5 to 1.1, e.g., 1.4 to 1.1, e.g., 1.3 to 1.1, e.g., 1.2 to 1.1; less than 70%, 60%, 50%, 45%, 40%, 35%, 30% of the chains present in the composition have a molecular weight greater than 7500 or 8000 Da; less than 40%, 35%, 30%, 25% of the chains present in the composition have a molecular weight less than 5500 or 5000 Da; and the composition comprises a mixture of ΔU and I/G structures at the non-reducing ends of the chains.

In one embodiment, about 15%, 20%, 25%, 30%, 35%, 45%, 50% of the chains in the LMWH composition have a ΔU at the non-reducing end. Preferably, about 15% to 50%, e.g., 15% to 35% of the chains, e.g., 20% to 35% of the chains in the composition have a ΔU at the non-reducing end.

In one embodiment, the LMWH composition comprises a higher level of ΔUH_{NAc,6S}GH_{NS,3S,6S} than enoxaparin, daltaparin and/or UFH, e.g., comprises about 5 to 15 mole %, e.g., 7 to 14 mole %, e.g., 9 to 12 mole %, e.g., as measured by CE.

In one embodiment, the LMWH composition has a calcium content less than 3%, 2.5%, 2%, 1.5%, 1.0%, and/or a sodium content less than 30%, 25%, 20%, 15%, 10%. In one embodiment, the LMWH composition comprises: less than 1000 ng/mg, 750 ng/mg, 500 ng/mg, 250 ng/mg of a heparinase enzyme, e.g., a heparinase enzyme described herein; less than 1.0%, 0.5%, 0.3% w/w methanol; less than 1.0%, 0.5%, 0.3%, 0.1% w/w ethanol; less than 2.0%, 1.75%, 1.25%, 1.0%, 0.5%, 0.3%, 0.15% chloride; less than 15%, 10%, 5%, 2.5% water by weight; less than 2000, 1500, 1000, 950, 900, 850, 800, 750, 700, 650, 600, 550, 500, 450, 400, 350, 300 ppm of free sulfate.

In one embodiment, the LMWH composition provides increased TFPI release as compared to enoxaparin. In one embodiment, the LMWH provides at least a 2, 3, 4, 5, 6, 7, 8, 9, 10, 15, 20, 30, 40 fold increase in TFPI release as compared to enoxaparin.

In one embodiment, the LMWH composition produced by a method disclosed herein has an intravenous half life of about 30 minutes to 3 hours, e.g., about 1 to 2 hours.

In one embodiment, the LMWH composition has a subcutaneous half life of about 30 minutes to 3.0 or 3.5 hours, e.g., about 1.5 to 2.5 hours, e.g., about 2 hours.

In one embodiment of any of the above aspects, the LMWH composition has one or more of the following characteristics:

the composition has substantially no (e.g., at least 85%, 90%, 95% or more of the chains do not have) modified reducing end structures; at least 60%, 70%, 80%, 85%, 90%, 95%, 99% of the chains of the composition have H_{NAc} at the reducing end; less than 90%, 95%, 98%, 99%, preferably none of the chains of the composition have a sulfated ΔU at the non-reducing end; and there is substantially no linkage region (e.g., less than 0.1% linkage region) present in the composition. In one embodiment, the LMWH composition has two, three or all of these characteristics.

In another embodiment, the LMWH composition produced by a method described herein has the following properties:
a weight average molecular weight of about 5000 to 9500 Da;
anti-IIa activity of about 50 to 300 IU/mg;
anti-IIa activity that is at least 50% neutralizable with protamine, e.g., as measured by ACT or aPTT;
ΔUH_{NAc,6S}GH_{NS,3S,6S} is 5 to 15% of the composition, preferably ΔUH_{NAc,6S}GH_{NS,3S,6S} at the non-reducing end of about 5 to 15% of the composition;
an average chain length of about 10 to 19 disaccharides;
an anti Xa to anti-IIa ratio of 3:1 or less;
the anti-Xa to anti-IIa ratio remains relatively constant over the course of an administration of the LMWH, e.g., the anti-Xa to anti-IIa ratio varies no more than about ±1.5, ±1, ±0.5, or ÷0.2, over a period of about 30, 60, 120, 180, 240, 300 minutes. For example, if an initial ratio of anti-Xa activity to anti-IIa activity is 2, then the ratio measured at a second time (e.g., 30, 60, 120, 180, 240, 300 minutes) after the initial administration will preferably be less than 3, and preferably at or around 2.

In a preferred embodiment, the LMWH composition produced by a method described herein has the following structure:

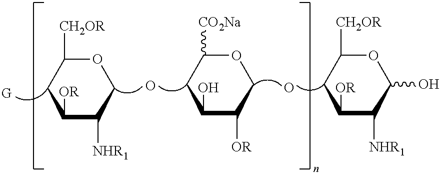

wherein:

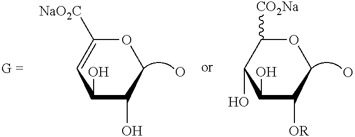

R is H or SO₃Na;

R1 is SO₃Na or COCH₃;

n=2-50, e.g., 2-40; and

the composition preferably has an average value for n of 10 to 19 and 5 to 15% of the chains in the composition have ΔUH_{NAc,6S}GH_{NS,3S,6S}, e.g., about 5 to 15% of the chains of the composition have ΔUH_{NAc,6S}GH_{NS,3S,6S} at the non-reducing end of the molecule, e.g., as measured by CE as a mole %.

In a preferred embodiment, the LMWH composition has the following properties:

anti-Xa activity of about 100 to 400 IU/mg;
anti-Xa activity that is at least 50% neutralizable, e.g., as measured by anti-Xa activity, ACT or aPTT;

a polydispersity of less than 1.6;

less than 70%, 60%, 50% of the chains present in the composition have a molecular weight greater than 7500 Da;

less than 40% of the chains present in the composition have a molecular weight less than 5000 Da;

it includes a mixture of ΔU and I/G structures at the non-reducing ends of the chains;

it has substantially no modified reducing end structures; at least 60%, 70%, 80%, 90% of the chains of the composition have HNAc at the reducing end;

about 15% to 35% of the chains in the composition have a ΔU at the non-reducing end;

less than 90%, 95%, 98%, 99%, preferably none of the chains of the composition have a sulfated ΔU at the non-reducing end.

In a preferred embodiment, the LMWH composition has the following properties:

it has a higher level of ΔUH_{NAc,6S}GH_{NS,3S,6S} than enoxaparin, daltaparin and/or UFH, e.g., ΔUH_{NAc,6S}GH_{NS,3S,6S} is present at about 5 to 15 mole %, e.g., as measured by CE.

In a preferred embodiment, the LMWH composition has the following properties:

it has a calcium content less than 3% and/or a sodium content less than 20%;

it includes less than 1000 ng/mg of a heparinase enzyme;

it has less than 1.0% w/w methanol;

it has less than 1.0% w/w ethanol;

it has less than 2.0% chloride;

it has less than 15% water by weight;

it has less than 2000 ppm of free sulfate.

In a preferred embodiment, the LMWH composition has the following properties:

it provides increased tissue factor pathway inhibitor (TFPI) release as compared to enoxaparin.

In a preferred embodiment, the LMWH composition has an intravenous half life of about 30 minutes to 3 hours.

In another aspect, the invention features a LMWH composition made by any of the methods described herein.

In some embodiments, the LMWH composition is dried. For example, the LMWH compositions may be dried by lyophilization, spray drying, or a drum dryer operation. In other embodiments, the LMWH composition is a liquid.

Other features and advantages of the invention will be apparent from the following detailed description, and from the claims.

### DESCRIPTION OF THE DRAWINGS

FIG. 1 presents a flow chart depicting the steps of a manufacturing process of LMWH compositions as described in an accompanying Example.

FIGS. 2A-2B present flow charts depicting comparative manufacturing processes of LMWH compositions as described in an accompanying Example.

### DETAILED DESCRIPTION

### Optimized LMWHs

The invention features methods of making LMWH compositions with the characteristics described herein at higher yields than previously achievable. For example, the methods described herein produce LMWH compositions with yields of at least about 10%, for example, 15%, 20%, 30%, 40%, 50%, 60%, 70%, 80%, 90% or more. Such methods can be used to make LMWH compositions designed to have properties that are clinically advantageous, e.g., over other commercially available LMWH preparations and UFH preparations. Such properties include, e.g., one or more of: reversibility with proteomine sulfate; predictable pharmacokinetics, anti-IIa activity; substantially constant anti-Xa activity to anti-IIa activity ratio; monitorable activity levels by standard tests such as, e.g., ACT or aPTT; subcutaneous bioavailability; and reduced occurrence of HIT.

Anti-IIa Activity

LMWH compositions are disclosed herein that include a significant number of chains of sufficient length (which can be described, e.g., in terms of average chain length of the preparation and/or weight average molecular weight of the preparation) to provide anti-IIa activity, e.g., anti-IIa activity of about 50 to 300 IU/mg, about 70 to 280 IU/mg, about 90 to 250 IU/mg, about 100 to 140 IU/mg, about 100 to 140 IU/mg, about 150 to about 200 IU/mg, about 130 to 190 IU/mg, about 155 to 195 IU/mg. Anti-IIa activity is calculated in International Units of anti-IIa activity per milligram using the statistical methods for parallel line assays. The anti-IIa activity levels described herein are measured using the following principle.

M118+ATIII→[M118·ATIII]IIa

M118·ATIII→[M118·ATIII·IIa]+IIa(Excess)

IIa(Excess)+Substrate→Peptide+pNA (measured spectrophotometrically)

Anti-factor IIa activity is determined by the sample potentiating effect on antithrombin (ATIII) in the inhibition of thrombin. Thrombin excess can be indirectly spectrophotometrically measured. The anti-factor IIa activity can be measured, e.g., on a Diagnostica Stago analyzer or on an ACL Futura3 Coagulation system, with reagents from Chromogenix (S-2238 substrate, Thrombin (53nkat/vial), and Antithrombin), or on any equivalent system. Analyzer response is calibrated using the 2nd International Standard for Low Molecular Weight Heparin.

Chain Length/Molecular Weight

A determination of whether a LMWH composition includes chains of sufficient chain length can be made, for example, by determining the average chain length of the chains in the LMWH composition and/or by determining the weight average molecular weight of chains within the LMWH composition. When average chain length is determined, an average chain length of about 10 to 19 disaccharide repeats, indicates that a significant number of chains in the LMWH composition are of sufficient chain length.

“Average chain length” as used herein refers to the average chain length of uronic acid/hexosamine disaccharide repeats that occur within a chain. The presence of non-uronic acid and/or non-hexosamine building blocks (e.g., attached PEG moieties) are not included in determining the average chain length. Average chain length is determined by dividing the number average molecular weight (Mn) by the number average molecular weight for a disaccharide (500 Da). Methods of determining number average molecular weight are described below using SEC MALS.

Examples of such LMWH composition include the following:

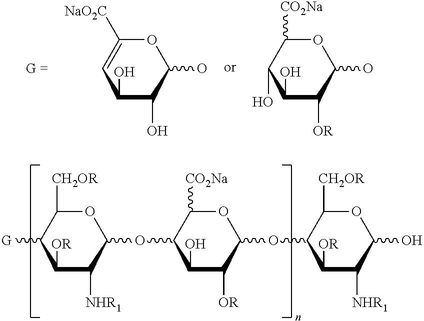

wherein R is H or SO₃X;
R1 is SO₃X or COCH₃ and X is a monovalent or divalent cation (e.g., Na or Ca);
and average n is about 10 to 19; and

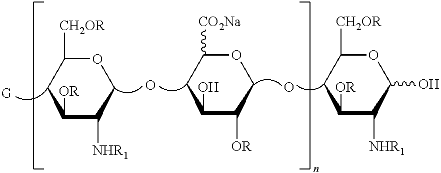

wherein

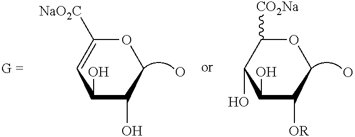

### R is H or SO₃X;

R1 is SO₃X or COCH₃, X is a monovalent or divalent cation (e.g., Na or Ca);
and average n is about 10 to 19.

When weight average molecular weight of a composition is determined, a weight average molecular weight of about 5000 to 9500 Da, about 5000 to 8300 Da, preferably about 5500 to 8000 Da, about 5700 to 7900, or about 5800 to 6800 Da, indicates that a significant number of chains in the LMWH composition are of sufficient chain length.

“Weight average molecular weight” as used herein refers to the weight average in daltons of chains of uronic acid/hexosamine disaccharide repeats. The presence of non-uronic acid and/or non-hexosamine building blocks are not included in determining the weight average molecular weight. Thus, the molecular weight of non-uronic acid and non-hexosamine building blocks within a chain or chains in the composition should not be included in determining the weight average molecular weight. The weight average molecular weight (M_{w}) is calculated from the following equation: M_{w}=Σ(cᵢmᵢ)/Σcᵢ. The variable cᵢ is the concentration of the polymer in slice i and Mᵢ is the molecular weight of the polymer in slice i. The summations are taken over a chromatographic peak, which contains many slices of data. A slice of data can be pictured as a vertical line on a plot of chromatographic peak versus time. The elution peak can therefore be divided into many slices. The weight average molecular weight calculation is average dependant on the summation of all slices of the concentration and molecular weight. The weight average molar weight can be measured, e.g., using the Wyatt Astra software or any appropriate software. The weight average molecular weights described herein are determined by high liquid chromatography with two columns in series, for example a TSK G3000 SWXL and a G2000 SWXL, coupled with a multi angle light scattering (MALS) detector and a refractometric detector in series. The eluent used is a 0.2 sodium sulfate, pH 5.0, and a flow rate of 0.5 mL/min.

Non-Reducing End Structure

In addition to chain length about 5 to 15 mole %, 7 to 14 mole %, or 9 to 12 mole % of the chains in a composition can have ΔUH_{NAc,6S}GH_{NS,3S,6S} at, or within about two, four or six monosaccharides from the non-reducing end of the chain. Methods that can be used to quantify this structure include, e.g., capillary electrophoresis (CE) and high performance liquid chromatography (HPLC), e.g., reverse phase high performance liquid chromatography (RPHPLC). To quantify the mole % of ΔUH_{NAc,6S}GH_{NS,3S,6S} in a LMWH composition, a response factor (RF) for ΔUH_{NAc,6S}GH_{NS,3S,6S} can be determined. The determination can also include determining the RF for all species obtained, e.g., using CE or HPLC, e.g., a CE method described herein. To obtain the RF for a species or all species obtained by CE, e.g., a CE method described herein, known concentrations of a standard for the specie or one or more of the species can be injected on the CE and used to determine a RF for each. The RF can then be used to determine the mole %. As described herein, the sample has an actual level of a structure, which can be expressed, e.g., as 5 to 15 when described in units of mole %. That actual level can also be expressed in other units, e.g., weight %. That actual level is the same regardless of the units in which it is expressed. The specification of mole % in the method is merely to indicate the actual prevalence of the structure. The level of structure can be measured in terms of other units and the reference value can be expressed in terms of other units, as long as the reference value as expressed in terms of alternative units corresponds to the same amount of structure as the reference value expressed in mole %, 5 to 15 mole % in this example. Thus, a method which requires showing the structure is present at 5 to 15 mole % can be performed by showing that the structure is present in a range expressed in an alternative unit of measure, e.g., weight %, chain number, or % AUC, wherein the range, as described in the alternative unit of measure, corresponds to the same amount of the structure which would give the mole % referred to, in this example 5 to 15 mole %.

A LMWH composition described herein can have a mixture of ΔU and iduronic acid (I)/glucuronic acid (G) at the non-reducing end of the chains in the composition. The nomenclature “−U” refers to an unsaturated uronic acid (iduronic acid (I), glucuronic acid (G) or galacturonic acid) that has a double bond introduced at the 4-5 position as a result, e.g., of the lyase action of a heparinase, a HSGAG lyase, or other enzyme having similar substrate specificity. Preferably, about 15% to 35%, 20 to 30% (e.g., 15%, 20%, 25%, 30%, 35%) of the total number of chains in the composition have a ΔU at the non-reducing end of the chain. The quantity of ΔU and/or I/G at the non-reducing end of chains within the sample can be determined using, e.g., 2D-NMR. In such methods, the total number of chains having an acetylated hexosamine (H_{NAc}) at the reducing end can be used to determine the total number of chains within the composition. The total percentage of chains having a ΔU and/or I/G at the non-reducing end can be compared to the total number of chains in the composition. Preferably, in the LMWH compositions described herein, less than 90%, 95%, 98%, 99% or none of the chains in the composition have a sulfated ΔU at the non-reducing end.

Reducing End Structures

In some instances, a LMWH composition provided herein has substantially no modified reducing end structures. In preferred embodiments at least 85%, 90%, 95%, 98%, 99% or all of the chains in the LMWH composition have a non-modified reducing end structure.

A “modified reducing end structure” refers to a structure that arises at the reducing end of chains in the composition due to the process of isolating or preparing the composition from natural sources. For example, many commercially available LMWH compositions are derived from unfractionated heparin primarily through chemical or enzymatic depolymerization of the polysaccharide chains. A process used to make a LMWH can cause one or more unique structural modifications to the reducing end of polysaccharide chains of starting material from a natural source. For example, nitrous acid depolymerization of heparin results in the formation of a 2,5-anhydromannose at the reducing end, which can be reduced to form an alcohol, and depolymerization through esterification of the carboxylate functional group on the uronic acid followed by β-elimination results in the formation of a 1,6-anhydro structures at the reducing end of some chains. Thus, 2,5-anhydromannose and 1,6 anhydro structures are examples of modified reduce end structures that can be found on some chains of LMWHs. The chains in a LMWH composition provided herein can include, e.g., at least about 60%, 70%, 80%, 85%, 90%, 95%, 98%, 99% or all of the chains having an acetylated hexosamine at the reducing end.

Anti-Xa Activity

Anti-Xa activity of a LMWH composition plays a role in biological activity of LMWH compositions. Preferably, a LMWH composition provided herein has an anti-Xa activity of about 100 to 400 IU/mg, e.g., about 120 to 380 IU/mg, e.g., about 150 to 350 IU/mg, e.g., about 170 to 330 IU/trig, e.g., about 180 to 300 IU/mg, e.g., about 150 to 200 IU/mg, 200 to 300 IU/mg. Anti-Xa activity of a LMWH composition is calculated in International Units of anti-factor Xa activity per milligram using the statistical methods for parallel line assays. The anti-factor Xa activity of LMWH compositions described herein is measured using the following principle:

M118+ATIII→[M118→ATIII]FXa

M118·ATIII→[M118·ATIII·FXa]+FXa(Excess)

FXa(Excess)+Substrate→Peptide+pNA (measured spectrophotometrically)

The anti-factor Xa activity is determined by the sample potentiating effect on antithrombin (ATIII) in the inhibition of activated Factor Xa (FXa). Factor Xa excess can be indirectly spectrophotometrically measured. Anti-factor Xa activity can be measured, e.g., on a Diagnostica Stago analyzer with the Stachrom® Heparin Test kit, on an ACL Futura3 Coagulation system with the Coatest® Heparin Kit from Chromogenix, or on any equivalent system. Analyzer response can be calibrated using the NIBSC International Standard for Low Molecular Weight Heparin.

Anti-Xa/IIa Ratio

In some aspects, LMWH compositions provided herein have an anti-Xa activity to anti-IIa activity ratio of 3:1 or less, e.g., 2:1, 1.6:1, 1.5:1, 1.4:1, 1.3:1, 1.2:1, 1.1:1, 1:1. Methods of determining anti-factor Xa activity and the anti-factor IIa activity have been described above. The ratio of anti-factor Xa activity to anti-factor IIa activity is calculated by dividing anti-factor Xa activity (dry basis) by the anti-factor IIa activity (dry basis).

Both anti-Xa activity and anti-IIa activity of heparin and LMWH compositions involve binding of antithrombin III (ATIII) to a specific sequence, represented by the structure ΔUH_{NAc,6S}GH_{NS,3S,6S}, within chains present in the composition. Binding of ATIII to this sequence mediates anti-Xa activity. In addition, thrombin (factor IIa) binds heparins at a site proximate to the ATIII binding site. Unlike anti-Xa activity that requires only the ATIII binding site, anti-IIa activity requires the presence of an ATIII binding site as well as a chain of sufficient length distal to the ATIII binding site. The anti-IIa activity of LMWH compositions provided herein can be attributed, at least in part, to the presence of ΔUH_{NAc,6S}GH_{NS,3S,6S} at or near the non-reducing end of chains within the LMWH compositions as well as the length of many of the chains present in the composition. This combination may result in chains within the composition that contribute to both anti-Xa activity and anti-IIa activity. When both anti-Xa activity and anti-IIa activity are provided by the same chain or chains, the clearance of that chain or chains can result in both a decrease in anti-Xa activity and anti-IIa activity. As such, the anti-Xa activity and anti-IIa activity can remain relatively constant over the course of administration. Therefore, in some aspects, the LMWH compositions provided herein have an anti-Xa activity to anti-IIa activity remains relatively constant over the course of an administration of LMWH, e.g., the anti-Xa activity to anti-IIa activity ratio varies about ±1.5, ±1, ±0.5, or ±0.2, over a period of about 30, 60, 120, 180, 240, 300 minutes. For example, if an initial ratio of anti-Xa activity t anti-IIa activity is 2, then the ratio measured at a second time (e.g., 30, 60, 120, 180, 240, 300 minutes) after the initial administration will preferably be less than 3, and preferably at or around 2.

Neutralization

LMWH compositions provided herein can be neutralized by protamine sulfate. For example, anti-Ha activity and/or anti-Xa activity can be neutralized by at least 50%, 60%, 70%, 80%, 85%, 90%, 95%, 99% or 100% by administration of protamine. Protamine sulfate is commercially available, e.g., from Eli Lilly and Company. Neutralization of anti-Xa activity and anti-IIa activity can be measured, e.g., by standard coagulation assays such as ACT and aPTT, both of which are described further herein. Protamine sulfate can be administered intravenously, e.g., at a dose of about 1, 2, 3 mg per 100 anti-Xa IU of the LMWH composition in plasma. Preferably, protamine neutralization of anti-Xa activity and/or anti-IIa activity occurs within 5, 10, 15, 20, 25, or 30 minutes after administration of the protamine sulfate.

Polydispersity

The polydispersity of LMWH compositions provided herein is about 1.6 or less, e.g., about 1.6 or 1.5 to 1.1, and numbers in between.

The term “polydisperse” or “polydispersity” refers to the weight average molecular weight of a composition (Mw) divided by the number average molecular weight (Mn). The number average molecular weight (Mn) is calculated from the following equation: Mn=Σcᵢ/(Σcᵢ/mᵢ). The variable cᵢ is the concentration of the polysaccharide in slice i and Mᵢ is the molecular weight of the polysaccharide in slice i. The summations are taken over a chromatographic peak, which contains many slices of data. A slice of data can be pictured as a vertical line on a plot of chromatographic peak versus time. The elution peak can therefore be divided into many slices. The number average molecular weight is a calculation dependent on the molecular weight and concentration at each slice of data. Methods of determining weight average molecular weight are described above, and were used to determine polydispersity as well.

For any of the ranges described herein, e.g., for a given structure or activity, the ranges can be those ranges disclosed as well as other ranges. For example, a range constructed from a lower endpoint of one range, e.g., for a given building block or activity, can be combined with the upper endpoint of another range, e.g., for the given building block or activity, to give a range.

An “isolated” or “purified” LMWH composition is substantially free of cellular material or other contaminating proteins from the cell or tissue source from which the LMWH is derived, or substantially free from chemical precursors or other chemicals when chemically synthesized. “Substantially free” means that a composition of LMWH is at least 50% pure (wt/wt). In a preferred embodiment, the composition of LMWH has less than about 30%, 20%, 10% and more preferably 5% (by dry weight), of non-heparin polysaccharides, proteins or chemical precursors or other chemicals, e.g., from manufacture. These also referred to herein as “contaminants”. Examples of contaminants that can be present in a LMWH composition provided herein include, but are not limited to, calcium, sodium, heparinase enzyme (or other enzyme having similar substrate specificity), methanol, ethanol, chloride, sulfate, dermatan sulfate, and chondrotin sulfate.

### Methods of Monitoring Activity of a LMWH Composition

The activity of a LMWH composition provided herein can be monitored by standard anti-coagulation assays. Such assays include, e.g., ACT and aPTT, both of which are routinely practiced in hospitals and specifically hospital operating rooms.

ACT is a test that is used to monitor the effectiveness of heparin therapy. The ACT can be done at the bedside, e.g., for patients experiencing pulmonary embolus, extracorporeal membrane oxygenation (ECMO) and hemodialysis. ACT is most often used before, during and after surgical intervention such as, e.g., cardiopulmonary bypass (CPB) surgery, PCI and stent placement. Reference value for the ACT can range from between 70-180 seconds. However, for certain procedures such as CPB the desired range can exceed 400-500 seconds. ACT utilizes negatively charged particles for a determination of time to clot formation. Examples of various particles that can be used include celite, which has a normal length of ACT being about 100 to 170 seconds; kaolin, which has a normal length of ACT being about 90 to 150 seconds; and glass particles, which have a normal length of ACT being about 190 to 300 seconds. Suitable machines for measuring ACT include, e.g., Hemochron and Medtronic HemoTec.

In the aPTT (also referred to as “partial thromboplastin time” or “PTT”) test, a contact activator is used to stimulate the production of Factor XIIa by providing a surface for the function of high molecular weight kininogen, kallikrein and Factor XIIa. This contact activation is allowed to proceed for a specific period of time. Calcium is then added to trigger further reactions and the time required for clot formation is measured. Phospholipids are required to form complexes, which activate Factor X and Prothrombin. APTT can be measured by the IL Test™ APTT-SP(liquid). Reference values for aPTT is about 25 to 35 seconds. A prolonged aPTT indicates that clotting is taking longer than expected, e.g., due to a heparin or LMWH treatment.

### Methods of Making LMWH Compositions

Various methods of making a LMWH composition are envisioned. Such methods result in higher yields of LMWH compositions than previously achievable. For example, the methods described herein produce yields of LMWH compositions of at least about 10%, for example, 15%, 20%, 30%, 40%, 50%, 60%, 70%, 80%, 90% or more. The term “yield” is used herein to refer to an amount of LMWH composition produced relative to an amount of a precursor material provided, for example, UFH composition. In some aspects as described herein, the methods result in yields that are at least about two-fold, three-fold, four-fold, five-fold, ten-fold, fifteen-fold, twenty-fold greater than that of methods that comprise fractionation of the UFH composition prior to digestion with the same enzyme and/or chemical.

In some embodiments of the production method, an UFH composition or other precursor may be provided. A precursor LMWH composition used in this method can be obtained by a method that includes enzymatic digestion of UFH starting material, e.g., an UFH that has not been fractionated. Preferably, the precursor has an average chain length of about 10 to 22, e.g., 10 to 20 disaccharides. For example, the precursor LMWH composition can have the following structure:

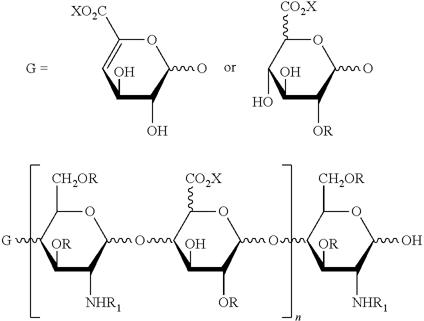

wherein X is a monovalent or divalent cation (e.g., Na or Ca),

### R is H or SO₃X;

### R1 is SO₃X or COCH₃;

n=2-45, e.g., 2-35;
and the composition preferably has an average value for n of 10 to 22, e.g., 10 to 20 disaccharides.

The UFH composition can be digested with an enzyme and/or chemical generally capable of cleaving glycosidic linkages. Such cleavage can occur for a period sufficient to provide a LMWH composition having a weight average molecular weight of about 5000 to about 11000 Da, e.g., about 5000 to 10000 Da. Other reactants or compositions can be present or added to the digestion mixture in addition to the enzyme and/or chemical.

Enzymatic digestion can include the use of one or more enzymes that cleaves at one or more glycosidic linkages of unsulfated uronic acids. Exemplary enzymes include heparinase III, mutants of heparinase III, heparinase II, mutants of heparinase II and HSGAG lyase III from Bacteroides thetaiotaomicron. Heparinase II and III are described, for example, in U.S. Pat. Nos. 5,681,733 and 5,919,693. Mutants of heparinase III are described in U.S. Pat. No. 5,896,789. Preferred heparinase III mutants are those mutants having one or more histidine at His36, His105, His110, His139, His152, His225 (also referred to herein as “MO11”), His234, His424, His469 and His539 substituted with an alanine. In at least one embodiment, the heparinase III enzyme may be present in a range of from about 25-500 units per gram of UFH, e.g., about 100-400 units per gram of UFH or 200-350 units per gram of UFH. For example, the heparinase III enzyme may be present at a concentration of about 300 units per gram of UFH.

Chemical digestion can be performed with a chemical or chemicals that cleave by oxidation (e.g., hydrogen peroxide), deamination (e.g., nitrous oxide) or beta elimination (e.g., quaternary ammonium).

Parameters and conditions relating to the digestion step may vary. In some embodiments, the UFH composition is digested at a temperature of about 25° C. to about 50° C. The unfractionated heparin composition may be digested at a pH level of about 5 to about 9. The UFH composition may be digested using a salt buffer. The buffer can be, e.g., a BisTris buffer, a phosphate buffer or an acetate buffer. For example, the salt buffer may be a monovalent or divalent salt buffer, e.g., a sodium acetate buffer. In at least some embodiments, the UFH composition is digested to completion, e.g., as indicated by a UV plateau, to provide the precursor LMWH composition.

In some embodiments of the method, the UFH composition is not size fractionated, e.g., precipitated, prior to digestion with the enzyme and/or chemical. In one embodiment, a yield of the purified LMWH composition produced by the method is at least about two-fold, three-fold, four-fold, five-fold, ten-fold, fifteen-fold, twenty-fold greater than that of a method that includes fractionation, e.g. precipitation, of the UFH composition prior to digestion with the same enzyme and/or chemical. For example, the yield may be at least about 2-, 3-, 5-, 10-, 15-, 20-, 30-, 40-, 50-, 60-, 70- or 80-fold greater than that of a method that comprises fractionation of the UFH composition prior to digestion with the same enzyme and/or chemical.

The method further includes fractionating the precursor LMWH composition, e.g., by a size dependent or charge dependent separation. For example, a size exclusion step or charge exclusion step may comprise size exclusion chromatography, ion exchange chromatography and/or filtration. The LMWH composition obtained from this step generally have an average chain length of about 10 to 19 disaccharides. The digested and fractionated LMWH composition can be the drug substance, or it can be further processed to obtain the final product, e.g., drug substance or drug product. Further processing steps can be used prior to or after the size dependant or charge dependent separation, e.g., to obtain drug substance or drug product. In some embodiments, the method further includes subjecting the LMWH composition to one or more purification steps. For example, the LMWH composition can be subjected to a filtration operation. In some embodiments, the method can include drying the LMWH composition, for example, by lyophilization, spray drying, or a drum dryer operation. In some embodiments, the method can include reconstituting a dried LMWH composition to provide, e.g., a drug product.

The term “drug product” refers to a LMWH composition having the purity required for and being formulated for pharmaceutical use.

The term “drug substance” refers to a LMWH composition having the polysaccharide constituents for pharmaceutical use but is not necessarily in its final formulation and/or comprises one or more non-product contaminant (e.g., one or more inorganic product such as sulfate, chloride, protein contaminant, process by-product such as heparinase, calcium, sodium).

Enzymes used in the methods described herein can be evaluated for substrate specificity by the following steps: 1) functional screening of enzyme activity against two HSGAG substrates having different sulfation densities, e.g., heparin and heparan sulfate, whereby enzymes having a preference for heparan sulfate over heparin are selected; 2) fragment mapping of cleaved substrates from step 1 to assess substrate specificity; 3) cleavage of a LMWH such as M118-REH step 1 intermediate or dalteparin using the enzyme, followed by; 4) assessment of anti-Xa activity and anti-IIa activity of the cleaved substrate using an in vitro assay; and 5) assessment of molecular weight distribution (or average chain length) of cleaved substrate using gel permeation chromatography (GPC) and/or size exclusion chromatography interfaced with multi-angle light scattering (SEC-MALS).

Step 1 assesses an enzyme's ability to act as an HSGAG lyase identified by the ability to generate an unsaturated C4-C5 bond at non-reducing ends of cleavage products as well as the enzymes preference for undersulfated substrates such as heparan sulfate. Enzyme activity can be followed spectrophotometrically by monitoring UV absorbance at 232 nm. An absorbance at this wavelength indicates formation of unsaturated uronic acids at the non-reducing ends of the cleavage product. Enzyme activity can be monitored both kinetically (initial rate of product formation) and in terms of total product formation following exhaustive digestion (about 12 to 15 hours). Preferred enzymes can have about a two fold preference for heparan sulfate over heparin and greater than a two fold (e.g., a 3 to 5 fold) difference in total activity.

The second step assesses the cleavage specificity of the enzyme. Enzymes suitable for making the LMWH compositions described herein preferentially cleave undersulfated regions of heparin or heparan sulfate. If UFH is the substrate used, this preference is demonstrated by an obvious underdigestion of substrate (as indicated by the presence of longer oligosaccharides) with any disaccharides being produced having a low sulfate density. In contrast, when the substrate is heparan sulfate, digestion results in a greater number of disaccharides which indicates a higher cutting frequency.

The remaining steps 3-5 can be performed as described elsewhere herein.

### Methods of Evaluating or Processing LMWH Compositions

One or more analytical techniques may be used to determine whether LMWH compositions made by any of the methods described herein have one or more of the described characteristics. Various types of analysis may be implemented such as, for example, capillary electrophoresis and nuclear magnetic resonance spectroscopy (NMR). Examples of how such methods can be employed are described in U.S. Patent Application Publication No. 2007-0287683, which is hereby incorporated herein by reference in its entirety.

### Other Embodiments

This invention is further illustrated by the following examples that should not be construed as limiting. The contents of all references, patents and published patent applications cited throughout this application are incorporated herein by reference.

### EXAMPLES

### Methods of Manufacturing M118-REH

Manufacturing Process Overview of a LMWH Composition by a Method Described Herein

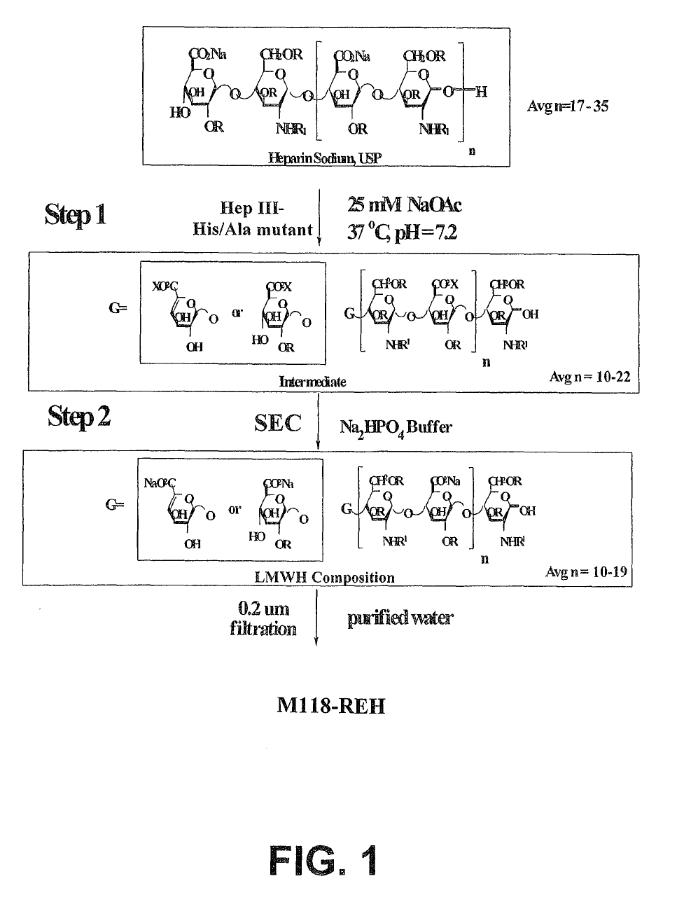

One embodiment of a method used to produce M118-REH at higher yields is shown in FIG. 1. Briefly, in Step 1 of the process, commercially available Unfractionated Heparin, USP (UFH) was subjected to a digestion process using a modified heparinase III enzyme having a substitution of an alanine for histidine at amino acid residue 225 (MO11) in aqueous sodium acetate buffer, pH 7.2 at 37° C. to produce Intermediate 2. MO11 cleaved by β-elimination between N-acetylglucosamine residues and under sulfated uronic acids producing chains having a Δ4,5 uronic acid group at the non-reducing end and an N-acetyl glucosamine at the reducing end. When digestion was complete, heat was turned off and sodium chloride was added to achieve a final solution concentration of approximately 2% w/v.

In Step 2, size exclusion chromatography (SEC) was used to remove higher molecular weight components of the Intermediate away from the materials having high anti-Xa and anti-IIa activity. The product of this step was designated the LMWH Composition.

Comparative Data

Fractionation of an UFH Sample Prior to Digestion

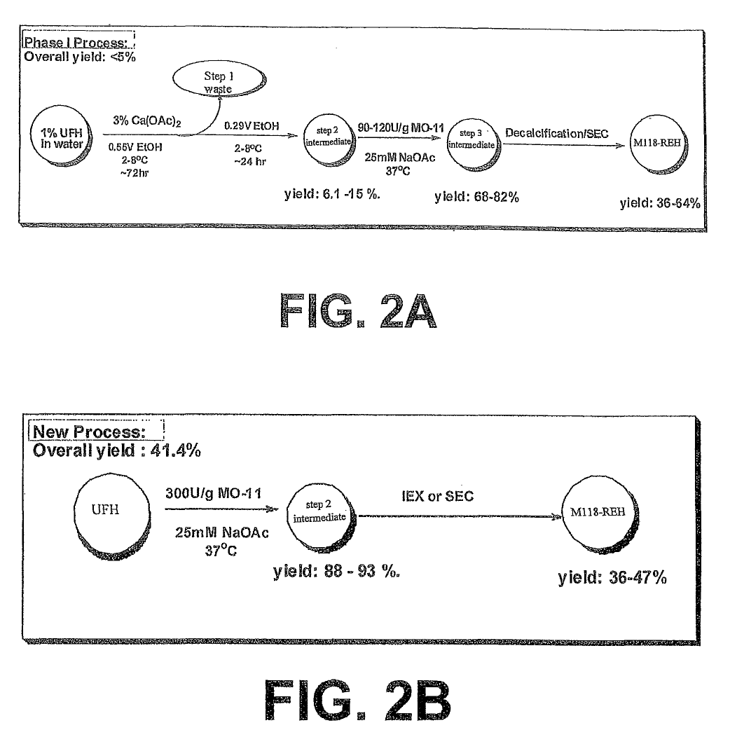

Per FIG. 2A, 1% wt/vol UFH, 3% wt/vol calcium acetate solution in water was sequentially precipitated using 0.55 volumes of Ethanol followed by a second precipitation with 0.29 volumes of ethanol. This step typically has a yield between 6.1-15%. The resulting step 2 intermediate was digested with MO11 enzyme and precipitated to afford the step 3 intermediate with yields between 68-82%. Step 3 intermediate was further decalcified and then purified via SEC column followed by selectively pooling and precipitating the combined pools to get crude M118-REH. This step has the typical yields ranging from 36-64%. Thus, the average overall process yields obtained with this process was less than 5%.

Direct Digestion of UFH Followed by Fractionation

Per FIG. 2B, UFH was directly digested with MO11 enzyme with an average digestion yield of 88-93%. This material (referred to as step 2) was directly taken to the SEC step and selectively pooled and precipitated to get the crude M118-REH with yields between 36 and 47%. Using this process, M118-REH that is consistent with the M118-REH produced via the FIG. 2A process was obtained with an overall process yield of 41.4%. This is approximately 10-fold higher than the FIG. 2A process yields.

What is claimed:

1. A method of making a low molecular weight heparin (LMWH) composition, comprising:
providing an unfractionated heparin composition;
digesting the unfractionated heparin composition with an enzyme, a chemical, or combinations thereof, that cleaves glycosidic linkages of unsulfated uronic acids to provide a precursor LMWH composition;
fractionating the precursor LMWH composition to provide the LMWH composition having a weight average molecular weight of about 5000 to about 9500 Da, an anti-IIa activity of about 50 to about 300 IU/mg,
wherein a yield of the LMWH composition produced by the method is at least about 10%.

2. The method of claim 1, wherein the unfractionated heparin is digested with an enzyme.

3. The method of claim 2, wherein the enzyme is heparinase III or a mutant of heparinase III having an alanine at amino acid residue 225 of the amino acid sequence of the mutant heparinase III.

4. The method of claim 2, wherein the enzyme is a heparin sulfate glycosaminoglycan lyase III from Bacteroides thetaiotaomicron.

5. The method of claim 1, further comprising filtering or precipitating the fractioned LMWH composition.

6. The method of claim 1, further comprising drying the fractionated LMWH composition.

7. The method of claim 1, wherein the fractionated LMWH composition has the following structure: 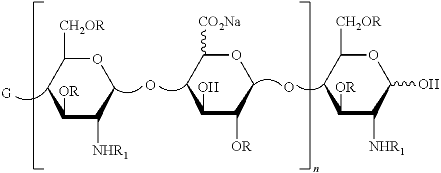
wherein: 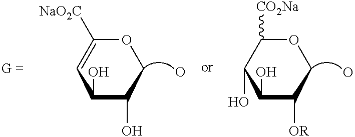
R is H or SO₃Na;
R1 is SO₃Na or COCH₃;
n=2-50;
wherein the composition has the following properties:
(a) an average value for n of 9-16,
(b) 5 to 15% ΔUH_{NAc,6S}GH_{NS,3S,6S} as measured by mole %,
(c) weight average molecular weight of about 5500 to 8500 Da,
(d) anti-Xa activity of about 100 to 400 IU/mg, and
(e) an anti-Xa to anti-IIa ratio of 2:1 to 1:1.

8. The method of claim 1, wherein the fractionated LMWH composition has an anti-IIa activity of about 50 to 300 IU/mg.

9. The method of claim 1, wherein the weight average molecular weight of the fractionated LMWH composition is about 5700 to 7900 Da.

10. The method of claim 1, wherein about 15 to 35% of the total number of chains in the fractionated LMWH composition have a ΔU at the non-reducing end.

11. The method of claim 1, wherein at least 60% of the chains in the fractionated LMWH composition have an N-acetylated hexosamine at the reducing end.

12. The method of claim 1, wherein at least 80% of the chains of the fractionated LMWH composition have an N-acetylated hexosamine at the reducing end.

13. A method of making a LMWH composition, comprising:
providing a precursor LMWH composition made by digesting an unfractionated heparin (UFH) composition with an enzyme, a chemical, or combinations thereof, that cleaves glycosidic linkages of unsulfated uronic acids; and,
subjecting the LMWH precursor composition to one or more fractionation steps to thereby make a fractionated LMWH composition, wherein the yield of the fractionated LMWH composition is at least about two-fold, three-fold, four-fold or five-fold greater than that of a method that includes precipitation of the UFH composition prior to digestion with the same enzyme, chemical or combinations thereof.
